(19) United States
(12) Patent Application Publication
(10) Pub. No.: US 2024/0312643 A1
(43) Pub. Date: Sep. 19, 2024

(54) APPARATUSES AND METHODS FOR TELESUPERVISION OF SERVICE PROVIDERS OF COUNSELING OR MEDICAL SERVICES

(71) Applicant: GET A-HEAD INC., Toronto (CA)

(72) Inventors: Ahad Karamali BANDEALY, Orlando, FL (US); Russell CHAN, Mono (CA)

(21) Appl. No.: 18/706,594

(22) PCT Filed: Nov. 3, 2022
(86) PCT No.: PCT/CA2022/051626

§ 371 Date: May 1, 2024

Related U.S. Application Data
(60) Provisional application No. 63/263,598, filed on Nov. 5, 2021.

(51) Int. Cl. G16H 80/00; G16H 40/20; H04L 12/18; H04L 51/046

(52) U.S. Cl. CPC G16H 80/00; G16H 40/20; H04L 12/1822; H04L 51/046

(57) ABSTRACT

A computer-implemented procedure for telesupervision including initially setting a visibility status of a supervisor computer as invisible to a service recipient computer for a service-providing audio/video conference: displaying, at a service provider computer, a service provider view of the conference: displaying, at a service recipient computer, a service recipient view of the conference; and displaying, at the supervisor computer, a supervisor view of the conference: wherein displaying the respective view of the conference at each respective computer comprises displaying either a real-time video feed from, or a participant indicator for, each and only each computer having a visibility status of visible to the respective computer.

### TECHNICAL FIELD

The disclosed subject matter relates to apparatuses and methods for telesupervision of a service provider of counseling services or medical services.

### BACKGROUND

An important factor and a requirement in the training of medical and counseling clinicians is the supervision of professional practice. Supervision is often regarded to serve three functions: one, normative: ensuring client well-being while observing and assessing the practitioner-in-training's competence: two, restorative: monitoring, supporting, and ensuring both professional and personal well-being of the trainee; and three, formative: cultivating and guiding the trainee's professional practice.

### SUMMARY

In some general aspects, a computer-implemented procedure for telesupervision is provided, including: initially setting a visibility status of a supervisor computer as invisible to a service recipient computer for a service-providing audio/video conference: displaying, at a service provider computer, a service provider view of the conference: displaying, at a service recipient computer, a service recipient view of the conference; and displaying, at the supervisor computer, a supervisor view of the conference: wherein displaying the respective view of the conference at each respective computer includes displaying either a real-time video feed from, or a participant indicator for, each and only each computer having a visibility status of visible to the respective computer.

Implementations can include one or more of the following. The procedure can also include initially setting a visibility status of a supervisor computer as invisible to a service provider computer for the conference. The procedure can also include providing a break-in input at the supervisor computer. The procedure can also include, after receiving a break-in input at the supervisor computer, setting the supervisor computer to visible at the service recipient computer. The procedure can also include, after receiving a break-in input at the supervisor computer, confirming the supervisor computer as visible at the service provider computer.

In additional general aspects, a computer-implemented procedure for telesupervision is provided, the procedure including, for a service-providing audio/video conference: initially setting a visibility status of a service provider computer as visible to all devices in the conference: initially setting a visibility status of a service recipient computer as visible to all devices in the conference: initially setting a visibility status of a supervisor computer as (1) invisible to the service recipient computer and, selectably by a user at the supervisor computer, (2) invisible to the service provider computer, in the conference: displaying, on a service provider graphical user interface displayed on a service provider screen at the service provider computer, a service provider conference display region including a service provider display of the conference: displaying, on a service recipient graphical user interface at the service recipient computer, a service recipient conference display region including a service recipient display of the conference: displaying, on a supervisor graphical user interface at the supervisor computer, a supervisor conference display region including a supervisor display of the conference; and providing at each respective computer, from each and only each computer having a visibility status of visible to the respective computer, an accessible real-time audio feed or a muted audio indicator: wherein displaying each respective display of the conference at each respective computer includes displaying either real-time video feed from, or a participant indicator for, each and only each computer having a visibility status of visible to the respective computer.

Implementations can include one or more of the following. The procedure can also include, after a visible-to-service-provider input has been provided at the supervisor computer, confirming the visibility status of the supervisor computer as visible to the service provider computer for the conference. The procedure can also include, after a break-in input is provided at the supervisor computer, changing the visibility status of the supervisor computer to visible to the service recipient computer for the conference. The procedure can also include, after the break-in input is provided at the supervisor computer, confirming the visibility status of the supervisor computer as visible to the service provider computer for the conference. The procedure can also include displaying, on the supervisor graphical user interface at the supervisor computer, a break-in device activatable by a user to provide the break-in input, and after the break-in input is provided, updating the break-in device.

The procedure can also include setting a conference control status of the service provider computer as host, and only if and after a break-in input has been provided at the supervisor computer, confirming a conference control status of the supervisor computer as host and changing the conference control status of the service provider computer to participant. The procedure can also include displaying, on the service provider graphical user interface at the service provider computer, a service provider first text chat display and entry region including a service provider display of a service text chat and displaying, on the service recipient graphical user interface at the service recipient computer, a service recipient text chat display and entry region including a service recipient display of the service text chat. The procedure can also include displaying, on the service provider graphical user interface at the service provider computer, a service provider second text chat display and entry region including a service provider display of a training text chat and displaying, on the supervisor graphical user interface at the supervisor computer, a training text chat display and entry region including a supervisor display of the training text chat. The procedure can also include provisioning the service text chat separately from the training text chat and using individual security tokens for each computer connected to the service text chat and for each computer connected to the training text chat.

In the procedure, each respective display of the service text chat can include respective lines of text chat entries with one or more pseudonymous identifiers of a service provider and/or a service recipient. In the procedure, each respective display of the training text chat can include respective lines of text chat entries with one or more pseudonymous identifiers of a service provider and/or a supervisor. The procedure can also include includes periodically checking visibility status for each computer and updating each respective display of the conference and each respective audio feed. The procedure can also include synchronizing the updating of each respective display of the conference and of each respective audio feed using peer-to-peer timing signals broadcast from each respective computer to each respective computer. The procedure can also include using an application programming interface (API) in implementing the procedure, with all API calls and API interfacing utilizing globally unique identifiers (globally unique IDs or GUIDs).

According to additional aspects, a method for telesupervision is provided, the method including: providing an audio/video conference room accessible at a service provider computer, a service recipient computer, and a supervisor computer by way of respective communication channels: with respect to the conference room: setting an initial visibility of the service provider computer as visible to all, setting an initial visibility of the service recipient computer as visible to all, and setting an initial visibility at the supervisor computer as invisible to the service recipient computer, and, selectably to a user at the supervisor computer, as invisible to the service provider computer: providing a user-activatable break-in device on the graphical user interface at the supervisor computer; and only in response to activation of the break-in device at the supervisor computer: setting the visibility at the supervisor computer to visible to the service recipient computer: confirming the visibility of the supervisor computer as visible to the service provider computer; and updating the break-in device at the supervisor graphical user interface.

Implementations can include one or more of the following. The method can include providing a training text chat room accessible only at the service provider computer and at the supervisor computer by way of respective communication channels. The method can include providing a service text chat room accessible at the service provider computer and at the service recipient computer by way of respective communication channels. The method can include validating a user at the service provider computer and validating a user at the service recipient computer before providing the conference room. The method can include giving each respective computer access to audio and/or video produced at each and only each computer having a visibility status of visible to the respective computer. The method can include periodically checking the visibility status of each respective computer and confirming the corresponding access at each respective computer to audio and video produced at each respective computer.

The method can include setting up a client-to-client broadcast communication from each to each of the service recipient computer, the service provider computer, and the supervisor computer. The method can include sending an instruction to one computer of the service provider, service recipient, and supervisor computers from a processor and from other computers of the of the service provider, service recipient, and supervisor computers, via the broadcast link or links, wherein the one computer executes the instruction on first receipt.

According to additional aspects, a telesupervision system is provided, the system including: a service provider computer having access to an audio/video conference room and having an initial visibility status in the conference room of visible to all devices in the conference room: a service recipient computer having access to the conference room and having an initial visibility status in the conference room of visible to all devices in the conference room: a supervisor computer having access to the conference room and having an initial visibility status in the conference room of (1) invisible to the service recipient computer and, if selected or not unselected by a user at the supervisor computer, (2) invisible to the service provider computer in the conference room: a service provider display of the conference room, within a service provider conference display region of a service provider graphical user interface on a service provider screen at the service provider computer, the display including either real-time video feed from, or a participant indicator for, each and only each computer having a visibility status of visible to the service provider computer in the conference room: a service recipient display of the conference room, within a service recipient conference display region of a service recipient graphical user interface on a service recipient screen at the service recipient computer, the display including either real-time video feed from, or a participant indicator for, each and only each computer having a visibility status of visible to the service recipient computer in the conference room: a supervisor display of the conference room, within a supervisor conference display region of a supervisor graphical user interface at the supervisor computer, the display including either real-time video feed from, or a participant indicator for, each and only each computer having a visibility status of visible to the supervisor computer in the conference room: a user-activatable break-in device at the supervisor computer configured when activated to set the visibility status of the supervisor computer in the conference room to visible to the service recipient computer and to confirm the visibility status of the supervisor computer in the conference room as visible to the service provider computer. In the system, the user-activatable break-in device at the supervisor computer can include an activatable device on the graphical user interface on the screen.

### DESCRIPTION OF DRAWINGS

FIG. 1A is a diagram of a computer having a screen with a graphical user interface displayed thereon, useful by a service provider in providing, and useful in the telesupervision of a service provider providing, counseling or medical services.

FIG. 1B is a diagram of the computer of FIG. 1A having a graphical user interface displayed on the screen thereof in which differs from FIG. 1A in that a real-time video from a supervisor computer is not shown on the graphical user interface of FIG. 1B.

FIG. 1C is a diagram of the computer of FIG. 1A having a different display on the graphical user interface thereof useful in group counseling and in the telesupervision of a service provider providing group counseling.

FIG. 2A is a diagram of a computer having a display with a graphical user interface displayed thereon, useful by a service recipient in receiving, and useful in the telesupervision of a service provider providing, counseling or medical services.

FIG. 2B is a diagram of the computer of FIG. 2A having a different display on the graphical user interface thereof, after a supervisor has “broken in” to the session.

FIG. 2C is a diagram of the computer of FIG. 2B having a different display on the graphical user interface thereof after a supervisor has “broken in” to the session.

FIG. 2D is a diagram of a computer having a display with a graphical user interface displayed thereon, useful by a service recipient in receiving, and useful in the telesupervision of a service provider providing group counseling.

FIG. 3A is a diagram of a computer having a display with a graphical user interface displayed thereon, useful by a supervisor in the telesupervision of a service provider providing counseling or medical services.

FIG. 3B is a diagram of the computer of FIG. 3A having a different display on the graphical user interface thereof, useful by a supervisor in the telesupervision of a service provider providing group counseling.

FIGS. 4A, 4B, 4C, and 4D are diagrams of various participant indicators such as can be used in the graphical user interfaces of FIGS. 1A-3B in place of an absent video feed.

FIGS. 5A and 5B are diagrams of examples video feeds with additional information included, such as can be used in the graphical user interfaces of FIGS. 1A-3B.

FIGS. 6 and 7 are flow diagrams of methods or processes according useful in the telesupervision of service providers of counselling or medical services.

FIG. 8 is a block diagram of various aspects of hardware and software useful for implementing the apparatuses and methods disclosed herein.

FIG. 9 is a block diagram of some steps of a method or process according to aspects of the present disclosure.

Further features and advantages of various embodiments, as well as the structure and operation of various embodiments, are described in detail below with reference to the accompanying drawings. It is noted that teachings contained herein are not limited to the specific embodiments described herein. Such embodiments are presented herein for illustrative purposes only. Additional embodiments will be apparent to persons skilled in the relevant art based on the teachings contained herein.

### DESCRIPTION

“Live supervision” is the practice of a supervisor being present and observing a medical student trainee's knowledge and communication skills with a patient, while directly intervening during a session to provide support and professional development. Live supervision is used in various settings including counseling skills programs, group counseling courses, student counseling programs, and post-graduate training, clinicians-in-training, and on their medical internships. As mentioned above, supervision is often regarded to serve three functions: one, normative: ensuring client well-being while observing and assessing the practitioner-in-training's competence: two, restorative: monitoring, supporting, and ensuring both professional and personal well-being of the trainee; and three, formative: cultivating and guiding the trainee's professional practice. The restorative and formative functions can be achieved, to some degree, at least, as live supervision is practiced today. Current live supervision practices, however, have not been shown to be effective in the normative function.

Live supervision has evolved through the years. Initially, it involved supervisors observing sessions through a physical one-way mirror. When communication needed to occur, supervisors would knock on the door, walk into the room, and speak to the supervisee face-to-face. To decrease intrusive supervisor-supervisee communication, a camera feed into an adjacent room could be set up, enabling the supervisor to provide guidance via telephone. A ringing telephone was still cumbersome and intrusive, so practitioners-in-training were later equipped with a “bug-in-the-ear” device so that the supervisor could provide guidance more unobtrusively.

The present disclosure provides, in effect, a digital one-way mirror. A clinician-in-training, referred to herein as a “service provider” can engage in a telehealth or tele-counseling session and his or her respective supervisor can join the session, unbeknownst to the client, and communicate back and forth with the trainee, providing clinical feedback. Furthermore, the supervisor can choose to “break into the call” and speak directly with the client if needed or desired. Finally the supervisor can, if desired, choose not to be seen by the service provider (trainee).

For the purposes of the present disclosure and the discussion below, the following terms are used with the following definitions.

Audio/video conference—an audio/video conference (or “conference” for short) in which a service, such as counselling or group counseling with a service provider and one or more service recipients, is or is to be delivered, in which participants, using a user interface such as a graphical user interface at a (at their respective) local participant computer, can both turn on and “mute” their audio and video feeds/inputs (their local microphone and camera feeds/inputs) and can enter and exit the conference.

User Computer—a user computer is any user-interfacing computer or other user-interfacing web- or cloud-connected or connectable device, such as a laptop, desktop, tablet computer, cell phone, tablet, including even internet connected user appliances such as a watch, glasses, or the like, as distinguished from computers such as servers, cloud computing capacity, and the like without direct user interfacing under normal circumstances.

Participant computer—in an audio/video conference, a participant computer is any user computer having access to the conference, including a user computer with an invisible visibility status.

Visibility status—a status identifier, for each of the participant computers in an audio/video conference, that controls and represents or can represent the visibility of local audio and video from the participant computer AND status/existence information of the participant computer in the conference. Invisible visibility status of a participant computer to a specified other participant computer or group of participant computers means neither the presence in the conference, nor the local audio or video if enabled, nor any other status of the invisible computer is presented/available at the specified other participant computer or group of participant computers in a display of the conference. Visible visibility status of a participant computer to a specified participant computer or group of participant computers means the presence of the computer in the conference, the local audio or video if enabled, and other basic status of the visible computer is presented/available at the specific participant computer or group of participant computers in a (respective) display of the conference. Visible to all visibility status of a participant computer means that the participant computer's existence and status in the conference, and local video and audio (if enabled), are presented/available at the other participant computers in their respective displays of the conference.

Confirming—confirming a status as X means either setting the status to X or checking whether the status is X and if not, changing the status to X.

Invisible-to-service-provider input—an input received/receivable at a supervisor computer to trigger confirming the visibility status of the supervisor computer as invisible to the service provider computer in an associated audio/video conference.

Visible-to-service-provider input—an input received/receivable at the supervisor computer to trigger confirming the visibility status of the supervisor computer as visible to the service provider computer in an associated audio/video conference.

Break-in input—an input received/receivable at the supervisor computer to trigger changing the visibility status of the supervisor computer to in an associated audio/video conference visible to the service recipient computer, and (in implementations) confirming the visibility status of the supervisor computer as visible to the service provide computer in the conference.

Conference control status—a status identifier, for each participant computer in an audio/video conference, that represents and controls the conference control inputs available at the respective participant computer (and thus available to the respective participant individual) in the conference. Host status means that one or more host controls, such as muting and unmuting input from one or more other computers in the conference, removing one or more other computers from the conference, are made available at the computer with host status. Participant status means that the host controls are not made available at the computer with participant status.

Service text chat—a text chat in which audio/video conference participant computers (a service provider computer and one or more service recipient computers) are connected and can take part (that is, the respective users at the respective computers can take part).

Training text chat—a text chat, separate from the service text chat, in which a service provider computer and a supervisor computer are connected and can take part (that is, the respective users at their respective computers can take part).

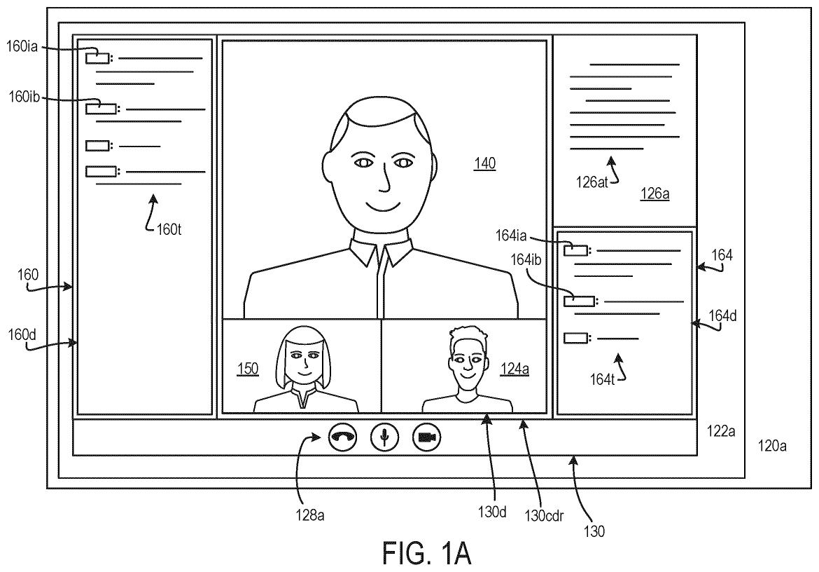

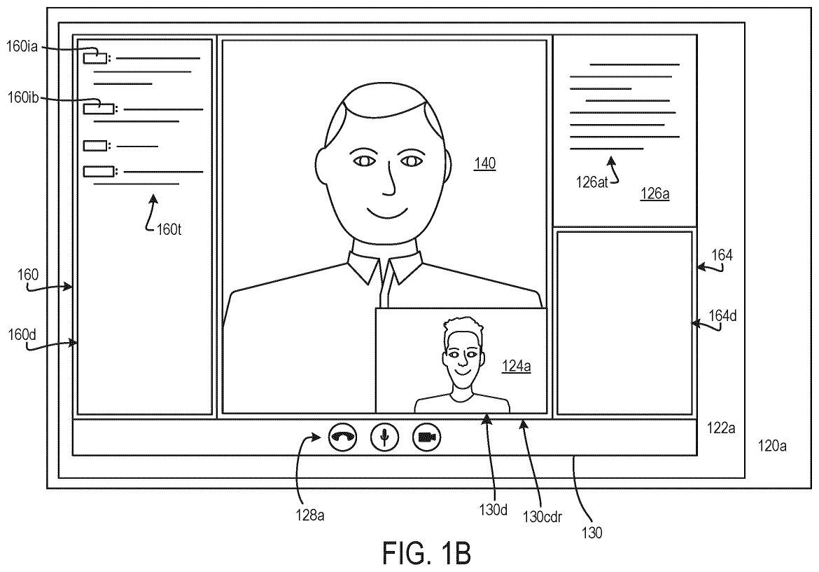

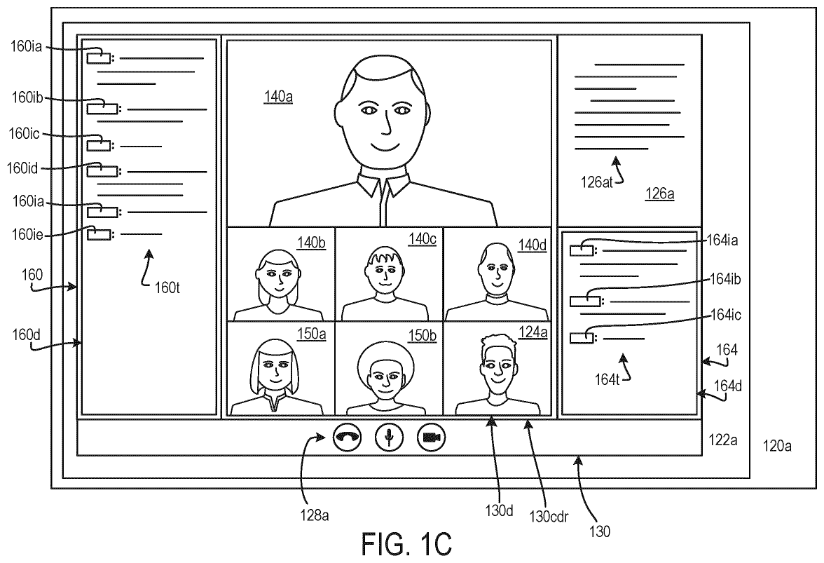

FIG. 1A is a diagram of a computer 120a having a display screen 122a with a graphical user interface 130 displayed thereon, useful by a service provider in providing, and useful in the telesupervision of a service provider providing, counseling or medical services. FIG. 1B is a diagram of the computer 120a of FIG. 1A having a graphical user interface 130 displayed on the display screen 122a thereof in which differs from FIG. 1A in that a real-time video from a supervisor computer, or other content representative of the presence of a supervisor computer, is not shown on the graphical user interface of FIG. 1B.

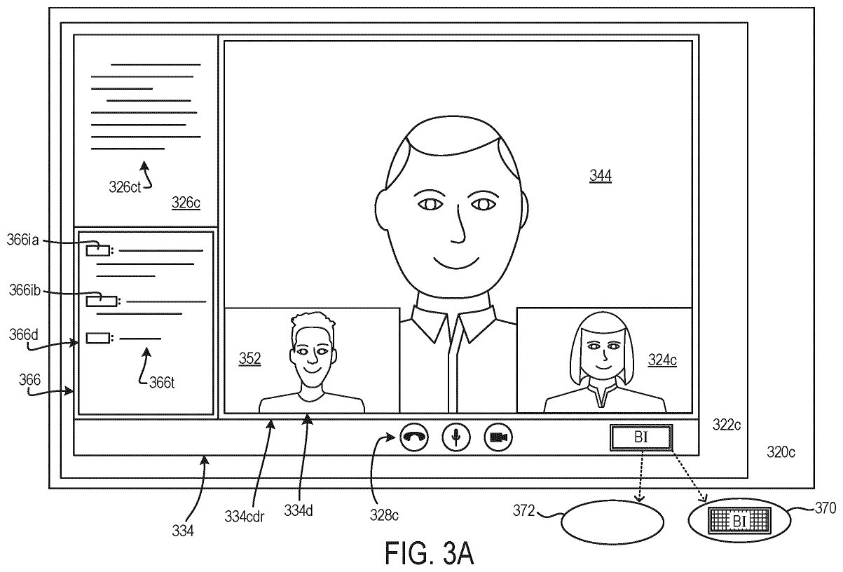

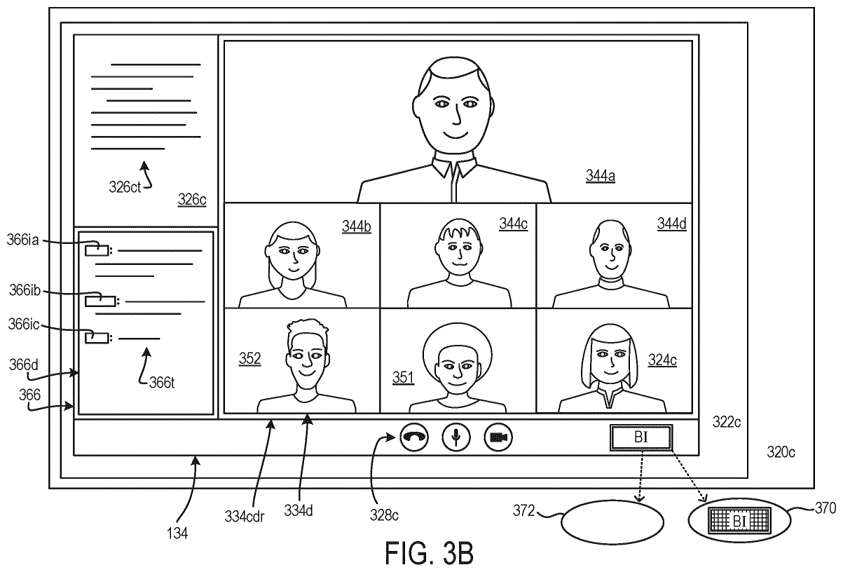

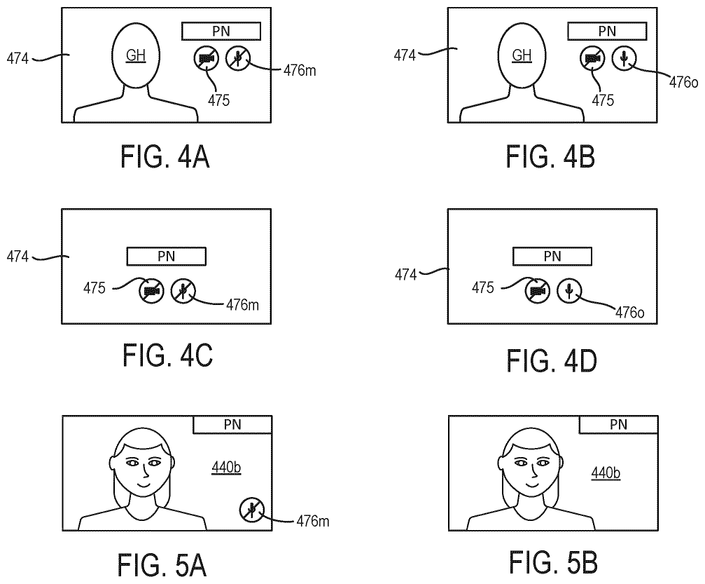

Referring to FIG. 1A, a diagram is shown of a computer 120a, such as a laptop or desktop computer, or a smart phone or tablet, or the like (all included within the term “computer” herein), having the display screen 122a with the graphical user interface 130 useful for a service provider, which can be referred to herein as a service provider graphical user interface 130, displayed thereon. The computer 120a can be referred to as a service provider computer 120a. The service provider graphical user interface 130 includes a conference display region 130cdr, which includes a service provider display 130d of, in this figure, a conference underway. In the example shown, the service provider display 130d of the conference includes a real-time video feed 140 from a service recipient computer, along with a real-time video feed 150 from a supervisor computer, and a real-time video feed 124a of a local camera built-in or connected to the service provider computer 120a (showing, in this case, a (local) video of a service provider). In the case of the service provider graphical user interface 130 shown in FIG. 1A, the supervisor computer (the supervisor computer is discussed below with respect to FIG. 3A) that provides the real-time video feed 150 has a visibility status of “visible” to the service provider computer 120a within the conference, and accordingly both (1) the real-time video feed 150 from the supervisor computer is present in the service provider display 130d of the conference (or, alternatively, in the absence of the video feed, a participant indicator for the supervisor computer), and (2) a real-time audio feed from the supervisor computer, synchronized to the real-time video feed is accessible at the service provider computer 120a (or, alternatively, a muted status indicator, see FIG. 4 below and the associated discussion, is present on the service provider display 130d of the conference). Referring to FIG. 1B, on the other hand, the supervisor computer can have a visibility status of “invisible” to the service provider computer 120a, in which case (1) the computer video feed from the supervisor computer (or a participant indicator for the supervisor computer) (video feed 150 in FIG. 1A) is not present in the service provider display 130d of the conference (or elsewhere at the service provider computer 120a), and (2) a real-time audio feed from the supervisor computer is not accessible at the service provider computer 120a and a muted indicator for the supervisor computer (see FIG. 4) also is not displayed on the service provider display 130d of the conference (or elsewhere at the service provider computer 120a). Also the second text chat display and entry region 164, in FIG. 1B, has either no service provider display 164d of a training text chat, or an empty service provider display 164d where the training text chat would be.

Referring FIGS. 1A and 1B, the graphical user interface 130 at the service provider computer 120a includes a first text chat display and entry region 160 having a service provider display 160d of a service text chat, and a second text chat display and entry region 164 having a service provider display 164d of a training text chat. A service provider and one or more service recipients can communicate via text messages using the service chat, while the service provider and one or more supervisors can communicate via text messages in the training chat. As shown in the figures, the chat displays 160d, 164d, can include lines of text 160t entered by one or more users of the service chat at one or more of the computers that are participants in the conference, and lines of text 164t as entered by one or more users at one or more of the service provider and supervisor computers connected to the training chat. In both chat displays, 160d and 164d, pseudonymous identifiers 160ia, 160ib, and 164ia, 164ib are used to identify the respective “speakers” in the text chat.

The service provider graphical user interface 130 at the service provider computer 120a can also include a notepad region 126a in which lines of text 126 at can be entered by the user to use as notes regarding a counseling session. The service provider graphical user interface 130 can also include conferencing controls 128a such as, for example, a call connect/disconnect control, a microphone on/mute control, a camera on/off control, and the like.

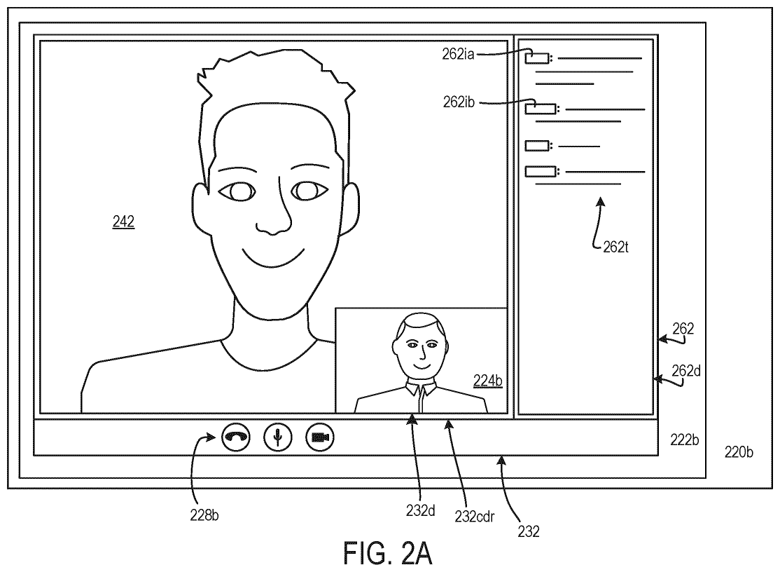

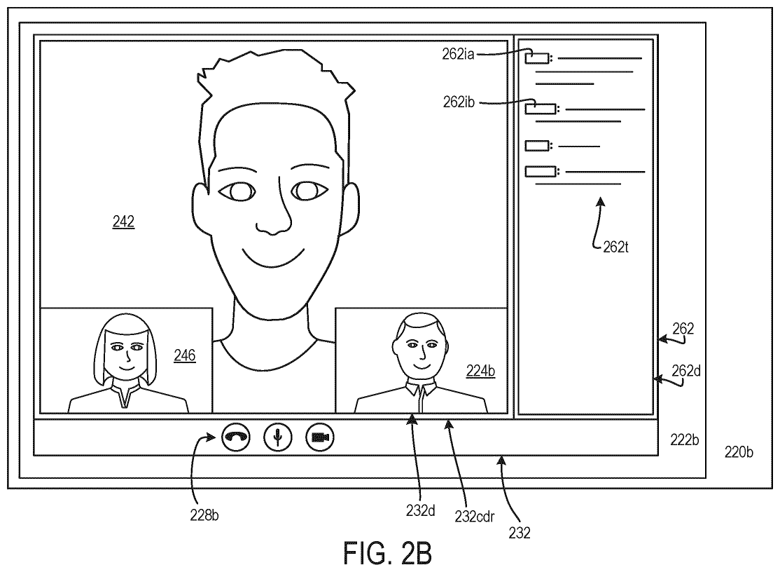

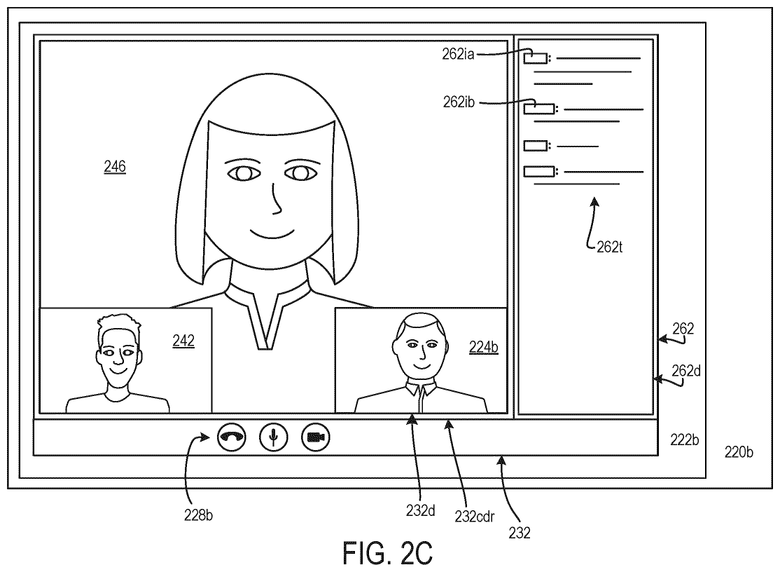

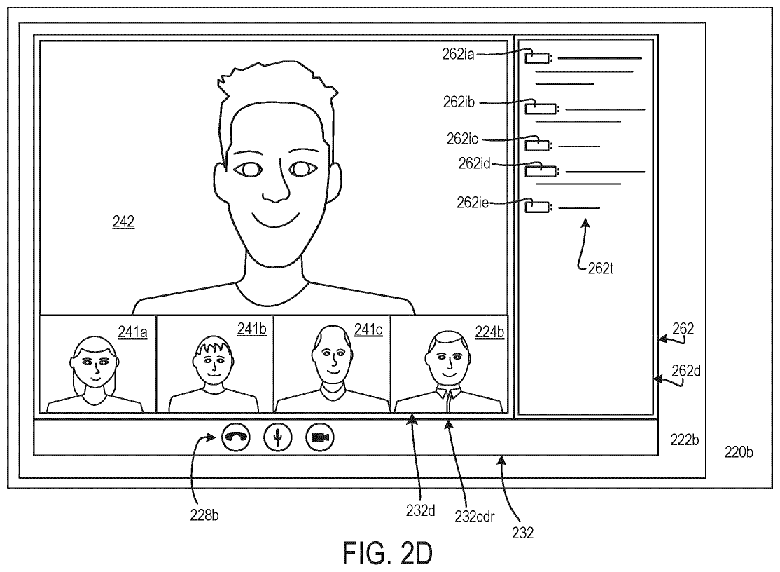

FIG. 2A is a diagram of a computer 220b useful for a service recipient, which can be referred to as a service recipient computer 220b. The service recipient computer 220b includes a service recipient display screen 222b, on which is displayed a service recipient graphical user interface 232. The service recipient graphical user interface 232 includes a service recipient conference display region 232cdr, which includes a service recipient display 232d of the conference of FIG. 1A. In implementation of the audio video conference shown in FIG. 2A, a supervisor computer (discussed below with respect to FIG. 3A) is a participant in the conference but has a visibility status of invisible to the service recipient computer 220b, so there is no video feed from, or participant indicator for, the supervisor computer included on the service recipient display 232d of the conference, and no audio feed from, or “muted” status indicator for, the supervisor computer is accessible at the service recipient computer 220b.

In contrast, the service provider computer 120a discussed above with respect to FIGS. 1A and 1B, also a participant in the conference, has a visibility status of visible to the service recipient computer 220b, or visible to all, so there is real-time video feed 242 from the supervisor computer 120a included in the service recipient display 232d of the conference, or in the absence of the video feed 242 (shown), a participant indicator for the service provider computer 120a (see FIG. 4 and the discussion of FIG. 4 below regarding participant and status indicators). A real-time audio feed from the supervisor computer, synchronized to the real-time video feed 242, is accessible at the service provider computer 120a or, alternatively, a “muted” status indicator (see FIG. 4 below and the associated discussion) is present on the service provider display 130d of the conference. A real-time video feed 224b of a local camera built-in or connected to the service recipient computer 220b is also included, showing, in this case, a (local) video of a service recipient.

As further shown in FIG. 2A, the graphical user interface 232 at the service recipient computer 220b includes a text chat display and entry region 262 including a service recipient display 262d of the service text chat mentioned above with respect to FIGS. 1A and 1B and defined above. The service provider user of the service provider computer 120a (FIGS. 1A, 1B) and the service recipient user of the service recipient computer 220b can communicate (along with any other service recipients, if present, from their respective service recipient computers) via text messages using the service chat. In the chat display 262d of the service chat, pseudonymous identifiers 262ia and 262ib (and others if more than two service recipient users/computers are present connected to the service chat) are used to identify different speakers in the service chat while preserving anonymity and associated confidentiality. The service recipient graphical user interface 232 can also include conferencing controls 228b such as, for example, a call connect/disconnect control, a microphone on/mute control, a camera on/off control, and the like.

FIG. 3A is diagram of a computer 320c useful by a supervisor in the telesupervision of a service provider providing counseling or medical services. The computer 320c can be referred to as a supervisor computer 320c. The supervisor computer 320c includes a display screen 322c with a supervisor graphical user interface 334 displayed thereon. The supervisor graphical user interface 334 includes a supervisor conference display region 334cdr having a supervisor display 334d of the conference. In the conference, the service provider computer 120a has a status of visible to the supervisor computer 320c or of visible to all, and the service recipient computer 220b also has a status of visible to the supervisor computer 320c or of visible to all. Accordingly, the supervisor display 334d of the conference includes a real-time video feed 344 from the service recipient computer 220b, along with a real-time video feed 352 from the supervisor computer 120a—or in the absence of a respective video feed, a respective participant indicator for the respective computer—and there is, accessible at the supervisor computer 320c, both a real-time audio feed from the service recipient computer 220b, synchronized to the real-time video feed (or a corresponding “muted” status indicator), and a real-time audio feed from the service provider computer 120a, synchronized to the real-time video feed 352 (or a corresponding “muted” status indicator). (See FIG. 4 and the associated discussion below regarding participant and status indicators).

Note that the relative sizes and positions of the feeds 344 and 352 in the supervisor display 334d of the conference are not particularly important. The two feeds 344 and 352 can be in various positions and can have various sizes, and these can be determined by input from a user at the supervisor computer, for example. The relative sizes and positions of the video feeds can also change automatically, for example, such as by placing the feed of the current or most recent speaker in the top and largest position (as shown for the feed 344 in FIG. 3A) with video feed of the non-current or less-recent speaker in a lower and smaller position, such as a picture-in-picture (PIP) position (as shown for the feed 352).

The supervisor graphical user interface 334 also includes a text chat display and entry region 366 including a supervisor display 366d of the training text chat, a text chat discussed above. A supervisor user of the supervisor computer 320c and a service provider user of the service provider computer 120a (FIGS. 1A, 1B) (and additional supervisors using additional supervisor computers, if any are participating in the conference) can communicate via text messages in the training chat, including while the conference is ongoing. As shown in FIG. 3A, the supervisor chat display 366d can include lines of text 366t entered by one or more users of the training chat at respective one or more computers connected to the training chat. Pseudonymous identifiers 366ia and 366ib (and more, if more than one supervisor user and supervisor computer are connected) are used to identify different speakers in the training chat while preserving anonymity and associated confidentiality.

The graphical user interface 334 at the service provider computer 320c can also include a notepad region 326c in which lines of text 326ct can be entered by the supervisor user to use as supervisor notes regarding a particular counseling and training session. The supervisor graphical user interface 334 can also include conferencing controls 328c such as, for example, a call connect/disconnect control, a microphone on/mute control, a camera on/off control, and the like.

When setting up or provisioning the conference, the visibility status of the service provider computer 120a (FIGS. 1A, 1B) is initially set as visible to all participant computers conference, the visibility status of the service recipient computer 220b (FIG. 2A) is also initial set as visible to all devices in the conference, and the visibility status of the supervisor computer 320c (FIG. 3A) is set as (1) invisible to the service recipient computer 220b, and, selectably by a user at the supervisor computer 320c, (2) invisible to the service provider computer.

In implementations, at any time during a conference, a user at the supervisor computer 320c can provide a visible-to-service-provider input, after which the system will confirm the visibility status of the supervisor computer 320c as visible to the service provider computer 120a for (the remainder of) the conference.

In implementations, a user at the supervisor computer 320c can provide a break-in input at the supervisor computer 320c that can cause the system to change the visibility status of the supervisor computer 320c to visible to the service recipient computer 220b for the (remainder of the) conference. The break-in input at the at the supervisor computer 320c can also cause the system to confirm the visibility status of the supervisor computer 320c as visible to the service provider computer 120a for (the remainder of) the conference, and to begin to display a training text chat in the display 164d of the second text chat display and entry region 164 at the computer 120a (see FIGS. 1A and 1B and the discussion of them above).

As also shown in FIG. 3A, in implementations, a break-in device BI can be included on the supervisor graphical user interface 334 at the supervisor computer 320c. The break-in device BI is activatable by a user (such as by a mouse-click or a touch on a touchscreen or the like) to provide the break-in input. After the break-in input is provided, the break-in device BI is updated, such as by highlighting it or changing its color as in inset 370 of FIG. 3A, or by stopping displaying the break-in device BI (thus removing it from the graphical user interface 334 or making it “disappear”) as in inset 372 of FIG. 3A.

According to another implementation, the procedure discussed can further include setting a conference control status of the service provider computer 120a (FIGS. 1A and 1B) as host, and only if and after a break-in input has been provided at the supervisor computer 320c (FIG. 3C), confirming a conference control status of the supervisor computer 320c as host and changing the conference control status of the service provider computer 120a to participant.

FIG. 2B is a diagram of the computer 220b of FIG. 2A, but having a slightly different display on the graphical user interface 232 thereof produced after a user at a supervisor computer participating in the conference (a supervisor) has provided a break-in input at the supervisor computer. As shown in FIG. 2B (by comparison with FIG. 2A), and as discussed above, after the break-in input has been provided at the supervisor computer (computer 320c of FIG. 3A), the visibility status of the supervisor computer 320c is set to visible to the service recipient computer 220b, and a real-time feed 246 from the supervisor computer 320c is therefore included in the service recipient computer display 232d of the conference. The real-time feed 246 may presented in relatively small format, such as in the PIP format of FIG. 2B, or may be presented in relatively large format, such as shown in the service recipient computer display 232d of the conference in FIG. 2C, for example. Alternatively, at the discretion of a user such as the user of the supervisor computer 320c, the format of the display 232d of the conference may adjust automatically, such as by putting the current or most recent speaker (of the service provider and the supervisor) at the top and in relatively larger format.

As mentioned above, pseudonymous identifiers can be used in each of the text chat displays 160d and 164d (FIGS. 1A, 1B), 262d (FIG. 2A), and 366d (FIG. 3A) to help protect the identities of users of the system and procedure. Pseudonymous identifiers can also be used in the process and system as a whole, including for example at an Application Programming Interface (API), so that identities are protected within the system as well. Also, globally unique identifiers (GUIDs) can be used for user computers and other system components and all API calls and interfacing. The use of GUIDs together with the use of pseudonymous identifiers helps ensure that that access to the system itself, whether by operators, or internet hosts of the system, or by some unauthorized path, does not result in easy identification of individual users. This helps protect the privacy of the participants in the chats (and in the conference) and the confidentiality of the communications made using the procedure and system of the present disclosure. The service text chat can also be provisioned separately from the training text chat, and individual security tokens can be used for each computer connecting to the service text chat and for each computer connecting to the training text chat, so that no crossover between the two text chats is possible. Additionally, visibility status for each computer 120a, 220b, 320c can be checked repeatedly and frequently, and each respective display 130d, 232d, 334d, of the conference, and the accessibility of each respective associated synchronized audio feed at each computer, can be changed or updated accordingly to match the current visibility statuses.

As mentioned, the process and system of the present disclosure can be implemented using an API. Because API calls and returns can sometimes have significantly varying time delays, the process and system can include establishing peer-to-peer broadcast links between the participating computers 120a, 220b, and 320c, and using broadcast timing signals among the computers 120a, 220b, and 320c, to synchronize changes to or updating of the respective computer display screens 122a, 222b, 322c or graphical user interfaces 130, 232, 334.

In additional implementations of the above-described processes and systems, multiple service recipients can be accommodated in the same conference, if desired, such as for a group therapy session or the like. Also, multiple supervisors may participate in supervision and/or training of the service provider.

For example, FIG. 1C is a is a diagram of the service provider computer 120a of FIG. 1A, but with multiple service recipient computers (four total, not shown) participating in the conference of FIG. 1C. Each of the multiple service recipient computers is providing a respective one of multiple service recipient computer real-time video feeds 140a, 140b, 140c, and 140d included in the service provider display 130d of the conference. Also in the example of FIG. 1C, first and second supervisor computers are participating in the conference, with corresponding first and second supervisor computer real-time video feeds 15a and 150b also included in the service provider display 130d of the conference.

In the first text chat display and entry region 160, the service text chat, as seen in the display 160d of the service text chat, includes more pseudonymous identifiers, namely, pseudonymous identifiers 160ia, 160ib, 160ic, 160id, and 160ie—one for the service provider (or service provider computer), plus one for each of the service recipients (or service recipient computers). In the second text chat display and entry region 164 where the display 164d of the training chat is shown, three pseudonymous identifiers, 164ia, 164ib, and 164ic, sufficient for the service provider and two supervisors are used.

FIG. 2D is a diagram of the service recipient computer 220b of FIG. 2A, with multiple, namely four service recipient computers participating the conference shown, such as the conference shown in FIG. 1C, in which four service recipient individuals participate group counseling together. So the service recipient computer 220b shown in FIG. 2D is one of four such computers participating in the conference. The three other service recipient computers (not shown) provide a respective one of multiple additional service recipient computer real-time video feeds, three in this case, namely, feeds 241a, 241b, and 241c. As in FIG. 2A, the participating service provider computer provides a service provider computer real-time video feed 242, and a local video feed 224b is also included in (or imposed on) the display 232d of the conference. The service text chat, as shown in the display 262d of the service text chat in the text chat display and entry region 262, employs five pseudonymous identifiers, 262ia, 262ib, 262ic, 262id, and 262ie, one for the service provider computer (or for the service provider user) and one for each of four service recipient computers (only one shown) (or service recipient users).

FIG. 3B is a diagram of the service recipient computer 320c of FIG. 3A, with multiple, namely four service recipient computers (with respective service recipient users) and two supervisor computers (with respective supervisor users) participating the conference. In this example, the four service recipient computers (one of which, computer 232b shown in FIG. 2D and discussed above) provide four real-time service recipient computer video feeds 344a, 344b, 344c, and 344d, and the two supervisor computers (one of which, computer 120a shown in FIG. 1C and discussed above) provide two supervisor computer real-time video feeds 351 and 352. As in FIG. 3A, a local video feed 324c is also included in (or imposed on) the display 334d of the conference. Similarly to FIG. 1C, a text chat display and entry region 366 includes a supervisor display 366d of the training text chat, a text chat which in this example employs three pseudonymous identifiers, 366ia, 366ib, and 366ic—one each for the service provider computer (and a service provider user) and two for the two supervisor computers (and corresponding two supervisor users).

FIGS. 4A, 4B, 4C, and 4D are diagrams of various participant indicators 474 including or containing status indicators. The participant indicators 474 can be used in the graphical user interfaces such as interfaces 130, 232, and 334 shown in FIGS. 1A-1C, 2A-2D, 3A, and 3B discussed above, such as in place of an absent video feed, as when a video feed from a participant computer is “muted” or turned off at the participant computer. With reference to FIGS. 4A through 4C, a participant indicator 174 replacing a video feed can include a video mute status indicator 475 and either a microphone mute status indicator 476m or a microphone on status indicator 4760. A pseudonymous identifier PN can also be included. A generic human profile or outline GH can also be included or omitted. Note that when a participant computer's visibility status is invisible at a given computer, no participant indicators and no status indicators are displayed at the given computer for that participant's computer. The participant indicators 174 and status indicators of FIGS. 4A-4D are used only to replace absent video feeds of visible computers.

FIGS. 5A and 5B show examples of a modified video feed 440b—examples of techniques that can be used for potentially any video feed of FIGS. 1A-1C, 2A-2D, 3A, and 3B discussed above, such as, for example, feed 140b of FIG. 1C. As shown in FIGS. 5A and 5B, a video feed 440b can include a microphone mute indicator 476m as in FIG. 5A when a microphone has been muted by a user at the source computer of the video feed. A microphone on indicator as in FIG. 4B could be used, or the video could be shown without any microphone indicator as in FIG. 5B, when the microphone is on. In either case, if desired, a pseudonymous identifier PN can also be included in or on the video feed 440b.

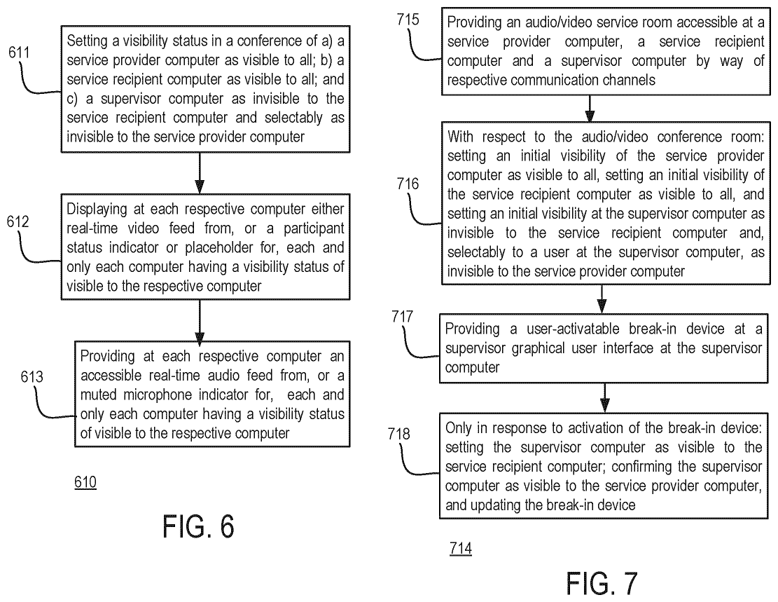

FIGS. 6 and 7 are flow diagrams showing main elements of methods or processes of the present disclosure.

As shown in FIG. 6, according to some aspects, a method or process 610 includes setting a visibility status in a conference of a) a service provider computer as visible to all: b) a service recipient computer as visible to all; and c) a supervisor computer as invisible to the service recipient computer and selectably as invisible to the service provider computer (611); displaying at each respective computer either real-time video feed from, or a participant indicator or placeholder for, each and only each computer having a visibility status of visible to the respective computer (612); and providing at each respective computer an accessible real-time audio feed from, or a muted microphone indicator for, each and only each computer having a visibility status of visible to the respective computer (613).

In another aspect related to the process or method of FIG. 6, a computer-implemented procedure for telesupervision is provided, with the procedure including, for a service-providing audio/video conference: initially setting a visibility status of a service provider computer 120a (FIGS. 1A, 1B) as visible to all computers in the conference: initially setting a visibility status of a service recipient computer 220b (FIG. 2A) as visible to all computers in the conference: initially setting a visibility status of a supervisor computer 320c (FIG. 3A) as (1) invisible to the service recipient computer 220b and, selectably by a user at the supervisor computer 320c, (2) invisible to the service provider computer 120a in the conference: displaying, on a service provider graphical user interface 130 displayed on a service provider screen 122a at the service provider computer 120a (FIGS. 1A, 1B), a service provider conference display region 130cdr including a service provider display 130d of the conference: displaying, on a service recipient graphical user interface 232 at the service recipient computer 220b, a service recipient conference display region 232cdr including a service recipient display 232d] of the conference (FIG. 2A): displaying, on a supervisor graphical user interface 334 at the supervisor computer 320c, a supervisor conference display region 334cdr including a supervisor display 334d of the conference; and providing at each respective computer 120a, 220b, 320c, from each and only each computer 120a, 220b, 320c having a visibility status of visible to the respective computer, an accessible real-time audio feed or a muted microphone indicator: wherein displaying each respective display 130d, 232d, 334d of the conference at each respective computer 120a, 220b, 320c includes displaying either real-time video feed from, or a participant indicator for each and only each computer (of 120a, 220b, and 320c) having a visibility status of visible to the respective computer (of 120a, 220b, and 320c).

As shown in FIG. 7, according to another aspect, a method or process 714 for telesupervision includes providing an audio/video conference room accessible at a service provider computer, a service recipient computer and a supervisor computer by way of respective communication channels (715): with respect to the conference room: setting an initial visibility of the service provider computer as visible to all, setting an initial visibility of the service recipient computer as visible to all, and setting an initial visibility at the supervisor computer as invisible to the service recipient computer and, selectably to a user at the supervisor computer, as invisible to the service provider computer (716): providing a user-activatable break-in device at a supervisor graphical user interface at the supervisor computer (717); and only in response to activation of the break-in device: setting the supervisor computer as visible to the service recipient computer: confirming the supervisor computer as visible to the service provider computer, and updating the break-in device (718).

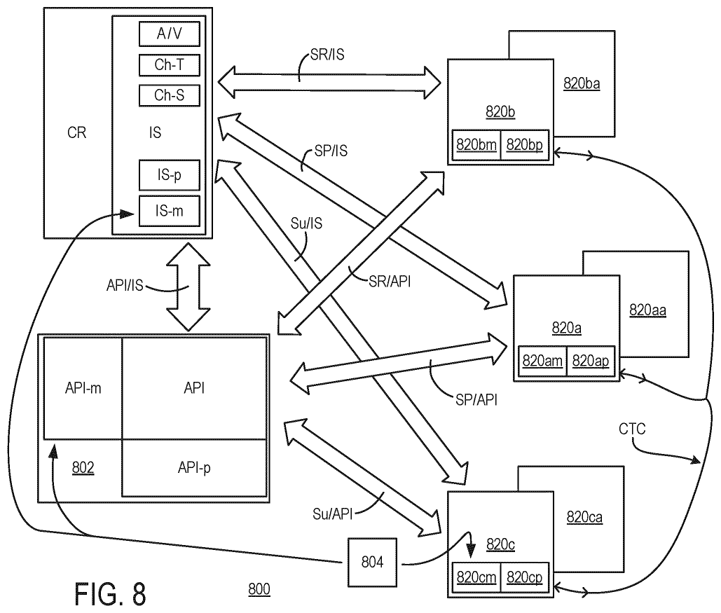

FIG. 8 is a diagram of various aspects of hardware and software useful for implementing the apparatuses and methods and systems disclosed herein. With reference to FIG. 8, a system 800 is used for the telesupervision of service providers of counseling or medical services and for providing the apparatuses and performing the processes and methods disclosed herein. The system 800 of FIG. 8 includes a service provider computer 820a, which can be a service provider computer such as service provider computer 120a in FIGS. 1A, 1B, and IC above. Similarly, the system 800 includes at least one service recipient computer 820b, which can be the computer 220b of FIGS. 2A through 2D, and at least one supervisor computer 820c, which can be the computer 320c of FIGS. 3A and 3B. The respective computers 820a, 820b, and 820c of FIG. 8 each need not be a single device or even a specific type of device. The system 800 can be controlled by an Application Programming Interface (API) that can allow multiple types of computers, such as a laptop or desktop computer, or a cell phone or tablet, or the like, as mentioned above, to serve as the respective computers 820a, 820b, and 820c, or also as respective alternate computers 820aa, 820ba, and 820ca, which can be used as a backup or reserve computers, or merely as alternative computers for convenience.

The API can reside in an environment 802 that can be a specific physical environment such as a secure server or a virtual secure environment. The API can include or have direct or indirect access to both an API processor or processing capacity API-p (which can be more than one physical o virtual processor) and to an API memory or API memory capacity API-m which can include both temporary and permanent storage (and which can be more than one physical or virtual memory). The API can interface with the computers 820a, 820b, and 820c and/or their local applications and one or more interaction services IS which may be hosted in one or more internet host service or cloud resource CR, allowing the API to provision and control various services or microservices in the interaction service IS and to control communications between services and the computers 820a, 820b, and 820c and between and among the computers 820a, 820b, and 820c. For the purposes of the system 800, a “processor” of the system can include not just the API processor API-p, but also internet service provider processing capability IS-p present in one or more internet or services ISA, and processing capability 820ap, 820bp, and 820cp present in the respective computers 820a, 820b, and 820c. Similarly, for the purposes of the system 300600, a “memory” of the system can include not just the API memory API-m, but also internet service memory IS-m present in one or more internet services IS, ISA, and memory 820 am, 820bm, and 820cm present in the respective computers 820a, 820b, and 820c. The “processor” of the API, as well as the individual processors 820ap 820bp, and 820cp of the individual computing devices 820a, 820b, and 820c can receive a computer program, for controlling the system and/or for implementing the methods and/or processes described herein, from one or more items of non-transitory computer readable storage media, such as non-transitory computer readable storage medium (or media) 804 which can reside in or be provided to the API directly or to any one or more of the computers 820a, 820b, 820c, such as to 820c as shown, and from one or more of the computing devices 820a, 820b, and 820c to the API (at least in part), or by other routes as desired.

Communication links SR/API, SP/API and Su/API can be established between the API and the service recipient, service provider, and supervisor computers 820, 820b, and 820c, respectively. Under the control of the API, communication links SR/IS, SP/IS, and Su/IS can be established between the internet service IS and the service recipient, service provider, and supervisor computers 820, 820b, and 820c, respectively. A direct peer-to-peer or “client-to-client” broadcast link or links can be established between each pair of the computers 820a, 820b, and 820c, such as link CTC as shown.

With continued reference to FIG. 8, and with reference to FIGS. 1A through IC, 2A through 2D, 3A and 3B also, in yet another aspect, similar to that of FIG. 7, a method or process for telesupervision is provided, the method including providing an audio/video conference room A/V accessible at a service provider computer 120a, 820a, a service recipient computer 220b, 820b, and a supervisor computer 320c, 820c by way of respective communication channels SP/IS, SR/IS, Su/IS: with respect to the conference room A/V: setting an initial visibility of the service provider computer 120a, 820a as visible to all (that is, all computers in, or that are participants in, the conference room A/V), setting an initial visibility of the service recipient computer 220b, 820b as visible to all, and setting an initial visibility at the supervisor computer 320c, 820c as invisible to the service recipient computer 220b, 820b, and, selectably to a user at the supervisor computer 320c, 820c, as invisible to the service provider computer 120a, 820a: providing a user-activatable break-in device BI on the graphical user interface 334 at the supervisor computer 320c, 820c; and, only in response to activation of the break-in device BI at the supervisor computer 320c, 820c: setting the visibility at the supervisor computer 320c, 820c to visible to the service recipient computer 220b, 820b; confirming the visibility of the supervisor computer 320c, 820c as visible to the service provider computer 120a, 820a; and updating the break-in device BI at the supervisor graphical user interface 334.

Implementations can include one or more of the following. The method or process can include providing a training text chat room Ch-T accessible only at the service provider computer 120a, 820a and at the supervisor computer 320c, 820c by way of respective communication channels SP/IS, Su/IS. The method can include providing a service text chat room Ch-S accessible at the service provider computer 120a, 820a and at the service recipient computer 220b, 820b by way of respective communication channels SP/IS, SR/IS. The method can include validating a user at the service provider computer 120a, 820a and validating a user at the service recipient computer 220b, 820b before providing the conference room A/V. The method can include giving each respective computer 120a, 820a, 220b, 820b, 320c, 820c access to audio and/or video produced at each and only each computer having a visibility status of visible to the respective computer 120a, 820a, 220b, 820b, 320c, 820c. The method can include periodically checking the visibility status of each respective computer (120a, 820a, 220b, 820b, 320c, 820c) and confirming the corresponding access at each respective computer (120a, 820a, 220b, 820b, 320c, 820c) to audio and video produced at each respective computer (120a, 820a, 220b, 820b, 320c, 820c).

The method can include setting up a client-to-client broadcast communication from each to each of the service recipient computer 220b, 820b, the service provider computer 120a, 820a, and the supervisor computer 320c, 820c. The method can include sending an instruction to one computer of the service provider 120a, 820a, service recipient 220b, 820b, and supervisor 320c, 820c computers from a processor such as a processor of the API and from other computers of the of the service provider 120a, 820a, service recipient 220b, 820b, and supervisor 320c, 820c computers, via the broadcast link or links, wherein the one computer executes the instruction on first receipt.

In still other aspects, a telesupervision system includes a service provider computer 120a, 820a] having access to an audio/video conference room and having an initial visibility status in the conference room of visible to all computers in (that is, having access to) the conference room: a service recipient computer 220b, 820b having access to the conference room and having an initial visibility status in the conference room of visible to all computers in the conference room: a supervisor computer 320c, 820c having access to the conference room and having an initial visibility status in the conference room of (1) invisible to the service recipient computer 220b, 820b and, if selected or not unselected by a user at the supervisor computer 320c, 820c, (2) invisible to the service provider computer 120a, 820a in the conference room: a service provider display 130d of the conference room, within a service provider conference display region 130cdr of a service provider graphical user interface 130 on a service provider display screen 122a at the service provider computer 120a, 820a, the display 130d including either real-time video feed from, or a participant indicator for, each and only each computer having a visibility status of visible to the service provider computer in the conference room: a service recipient display 232d of the conference room, within a service recipient conference display region 232cdr of a service recipient graphical user interface 232 on a service recipient display screen 222b at the service recipient computer 220b, 820b, the display 232d including either real-time video feed from, or a participant indicator for, each and only each computer having a visibility status of visible to the service recipient computer in the conference room: a supervisor display 334d of the conference room, within a supervisor conference display region 334cdr of a supervisor graphical user interface 334 at the supervisor computer 320c, 820c, the display 334d including either real-time video feed from, or a participant indicator for, each and only each computer having a visibility status of visible to the supervisor computer in the conference room: a user-activatable break-in device, the break-in device configured to set the visibility status of the supervisor computer 320c, 820c in the conference room to visible to the service recipient computer 220b, 820b and to confirm the visibility status of the supervisor computer 320c, 820c in the conference room as visible to the service provider computer 120a, 820a when selected or activated by a user. The break-in device can be an icon or button or the like BI on the graphical user interface 334 on the display screen 322c at the supervisor computer 320c, 820c.

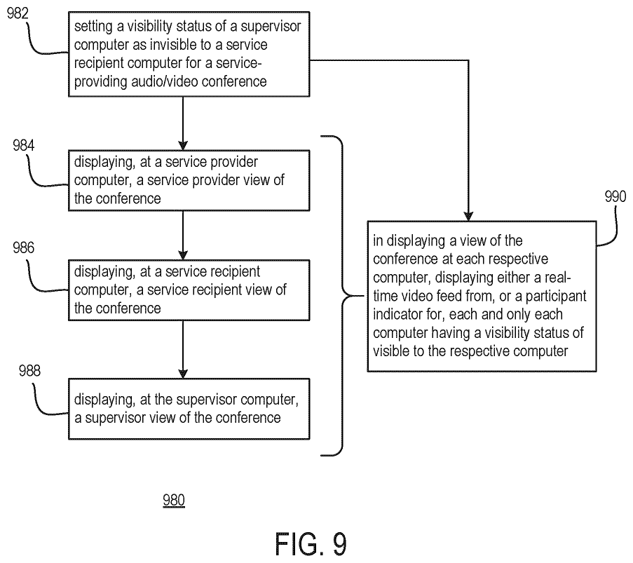

FIG. 9 is a block diagram of some steps of a method or process 980 according to aspects of the present disclosure. A procedure 980 for telesupervision diagrammed in FIG. 9 includes setting a visibility status of a supervisor computer as invisible to a service recipient computer for a service-providing audio/video conference (982) and while, as part of displaying a view of the conference at each respective computer, displaying either a real-time video feed from, or a participant indicator for, each and only each computer in the conference having a visibility status of visible to the respective computer (990), displaying, at a service provider computer, a service provider view of the conference (984): displaying, at a service recipient computer, a service recipient view of the conference (986); and displaying, at the supervisor computer, a supervisor view of the conference (988).

Using the methods, processes, and/or apparatuses of the present disclosure, communication between a supervisor (such as a supervisor user of the supervisor computer 320c of FIGS. 3A, 3B and/or 3C) and a service provider (such as a service provider user of the service provider computer 220b of FIGS. 2A and/or 2B through 2D) can occur during conferences (sessions) with minimal to no disruptions. Because supervision can occur silently during a counseling session or asynchronously via the training text chat displayed and entered on the second text chat display and entry region 164 of FIG. 1A of the service provider computer 120a and on the text chat display and entry region 366 of the supervisor computer 320c of FIG. 3A, for example, stronger rapport can be built between the service provider user of the service provider computer 120a of FIG. 1A and the service recipient user of the service recipient computer 220b of FIG. 2A. Conversational flow can be unencumbered, and autonomy of the service provider being trained can be cultivated as the service provider can decide how, when, and what feedback they will use. This has the potential to significantly help future clinicians as it encourages the development of clinical judgment. A major advantage of this technology is its potential to address normative functions (ensuring client well-being while observing and assessing the practitioner-in-training's competence), as evidence suggests a gap in both research and professional practice exists in achieving these functions. Service recipients, service providers, and supervisors can all use various computers, including smart phones, tablets, PCs, laptops, and so forth seamlessly, based on availability and their own comfort. All interactions occurring on the platform are private and securely stored, and can be collated for easy evaluation by the supervisor. Progress of a service provider student or trainee can be known and demonstrated easily, the quality of supervision can also be assessed.

Other implementations are within the scope of the following claims.

1. A computer-implemented procedure for telesupervision, the procedure comprising:
for a service-providing audio/video conference:
initially setting a visibility status of a service provider computer as visible to all devices in the conference;
initially setting a visibility status of a service recipient computer as visible to all devices in the conference;
initially setting a visibility status of a supervisor computer as (1) invisible to the service recipient computer and, selectably by a user at the supervisor computer, (2) invisible to the service provider computer, in the conference;
displaying, on a service provider graphical user interface displayed on a service provider screen at the service provider computer, a service provider conference display region including a service provider display of the conference;
displaying, on a service recipient graphical user interface at the service recipient computer, a service recipient conference display region including a service recipient display of the conference;
displaying, on a supervisor graphical user interface at the supervisor computer, a supervisor conference display region including a supervisor display of the conference; and
providing at each respective computer, from each and only each computer having a visibility status of visible to the respective computer, an accessible real-time audio feed or a muted audio indicator;
wherein displaying each respective display of the conference at each respective computer comprises displaying either real-time video feed from, or a participant indicator for, each and only each computer having a visibility status of visible to the respective computer.

2. The procedure of claim 1, further comprising at least one of:
after a visible-to-service-provider input has been provided at the supervisor computer, confirming the visibility status of the supervisor computer as visible to the service provider computer for the conference;
after a break-in input is provided at the supervisor computer, changing the visibility status of the supervisor computer to visible to the service recipient computer for the conference;
setting a conference control status of the service provider computer as host, and only if and after a break-in input has been provided at the supervisor computer, confirming a conference control status of the supervisor computer as host and changing the conference control status of the service provider computer to participant; or
using an application programming interface (API) in implementing the procedure, with all API calls and API interfacing utilizing globally unique identifiers (globally unique IDs or GUIDs).

3. (canceled)

4. (canceled)

5. The procedure of claim 2 further comprising at least one of:
after the break-in input is provided at the supervisor computer, confirming the visibility status of the supervisor computer as visible to the service provider computer for the conference; or
displaying, on the supervisor graphical user interface at the supervisor computer, a break-in device activatable by a user to provide the break-in input, and after the break-in input is provided, updating the break-in device.

6. (canceled)

7. (canceled)

8. The procedure of claim 1, further comprising displaying, on the service provider graphical user interface at the service provider computer, a service provider first text chat display and entry region including a service provider display of a service text chat and displaying, on the service recipient graphical user interface at the service recipient computer, a service recipient text chat display and entry region including a service recipient display of the service text chat.

9. The procedure of claim 8, further comprising at least one of:
displaying, on the service provider graphical user interface at the service provider computer, a service provider second text chat display and entry region including a service provider display of a training text chat and displaying, on the supervisor graphical user interface at the supervisor computer, a training text chat display and entry region including a supervisor display of the training text chat; or
wherein each respective display of the training text chat comprises respective lines of text chat entries with one or more pseudonymous identifiers of a service provider and/or a supervisor.

10. The procedure of claim 9, further comprising provisioning the service text chat separately from the training text chat and using individual security tokens for each computer connected to the service text chat and for each computer connected to the training text chat.

11. (canceled)

12. (canceled)

13. The procedure of claim 1 further comprising periodically checking visibility status for each computer and updating each respective display of the conference and each respective audio feed.

14. The procedure of claim 13 further comprising synchronizing the updating of each respective display of the conference and of each respective audio feed using peer-to-peer timing signals broadcast from each respective computer to each respective computer.

15. (canceled)

16. A method for telesupervision, the method comprising:
providing an audio/video conference room accessible at a service provider computer, a service recipient computer, and a supervisor computer by way of respective communication channels;
with respect to the conference room: setting an initial visibility of the service provider computer as visible to all, setting an initial visibility of the service recipient computer as visible to all, and setting an initial visibility at the supervisor computer as invisible to the service recipient computer, and, selectably to a user at the supervisor computer, as invisible to the service provider computer;
providing a user-activatable break-in device on the graphical user interface at the supervisor computer; and
only in response to activation of the break-in device at the supervisor computer:
setting the visibility at the supervisor computer to visible to the service recipient computer;
confirming the visibility of the supervisor computer as visible to the service provider computer; and
updating the break-in device at the supervisor graphical user interface.

17. The method of claim 16, further comprising at least one of:
providing a training text chat room accessible only at the service provider computer and at the supervisor computer by way of respective communication channels;
providing a service text chat room accessible at the service provider computer and at the service recipient computer by way of respective communication channels;
validating a user at the service provider computer and validating a user at the service recipient computer before providing the conference room; or
giving each respective computer access to audio and/or video produced at each and only each computer having a visibility status of visible to the respective computer.

18. (canceled)

19. (canceled)

20. The method of claim 16 further comprising giving each respective computer access to audio and/or video produced at each and only each computer having a visibility status of visible to the respective computer.

21. The method of claim 20, wherein giving each respective computer access to audio and/or video produced at each and only each computer having the visibility status of visible to the respective computer further comprises periodically checking the visibility status of each respective computer and confirming the corresponding access at each respective computer to audio and video produced at each respective computer.

22. The method of claim 21, further comprising setting up a client-to-client broadcast communication from each to each of the service recipient computer, the service provider computer, and the supervisor computer.

23. The method of claim 22, further comprising sending an instruction to one computer of the service provider, service recipient, and supervisor computers from a processor and from other computers of the of the service provider, service recipient, and supervisor computers, via the broadcast link or links, wherein the one computer executes the instruction on first receipt.

24. A telesupervision system comprising:
a service provider computer having access to an audio/video conference room and having an initial visibility status in the conference room of visible to all devices in the conference room;
a service recipient computer having access to the conference room and having an initial visibility status in the conference room of visible to all devices in the conference room;
a supervisor computer having access to the conference room and having an initial visibility status in the conference room of (1) invisible to the service recipient computer and, if selected or not unselected by a user at the supervisor computer, (2) invisible to the service provider computer in the conference room;
a service provider display of the conference room, within a service provider conference display region of a service provider graphical user interface on a service provider screen at the service provider computer, the display comprising either real-time video feed from, or a participant indicator for, each and only each computer having a visibility status of visible to the service provider computer in the conference room;
a service recipient display of the conference room, within a service recipient conference display region of a service recipient graphical user interface on a service recipient screen at the service recipient computer, the display comprising either real-time video feed from, or a participant indicator for, each and only each computer having a visibility status of visible to the service recipient computer in the conference room;
a supervisor display of the conference room, within a supervisor conference display region of a supervisor graphical user interface at the supervisor computer, the display comprising either real-time video feed from, or a participant indicator for, each and only each computer having a visibility status of visible to the supervisor computer in the conference room;
a user-activatable break-in device at the supervisor computer configured when activated to set the visibility status of the supervisor computer in the conference room to visible to the service recipient computer and to confirm the visibility status of the supervisor computer in the conference room as visible to the service provider computer.

25. The system of claim 24 wherein the user-activatable break-in device at the supervisor computer comprises an activatable device on the graphical user interface on the screen.

26. A computer-implemented procedure for telesupervision, the procedure comprising:
initially setting a visibility status of a supervisor computer as invisible to a service recipient computer for a service-providing audio/video conference; then:
displaying, at a service provider computer, a service provider view of the conference;
displaying, at a service recipient computer, a service recipient view of the conference; and
displaying, at the supervisor computer, a supervisor view of the conference;
wherein displaying the respective view of the conference at each respective computer comprises displaying either a real-time video feed from, or a participant indicator for, each and only each computer having a visibility status of visible to the respective computer.

27. The procedure of claim 26, further comprising at least one of:
initially setting a visibility status of a supervisor computer as invisible to a service provider computer for the conference; or
providing a break-in input at the supervisor computer.

28. (canceled)

29. The procedure of claim 27, wherein providing the break-in input at the supervisor computer further comprises, after receiving a break-in input at the supervisor computer, setting the supervisor computer to visible at the service recipient computer.

30. The procedure of claim 29 further comprising, after receiving a break-in input at the supervisor computer, confirming the supervisor computer as visible at the service provider computer.
